SEQUENCE LISTING (33 sequences)

SEQ ID NO: 1 - protein, 7 aa, synthetic construct
  source 1..7: note = synthetic
SNYLYDN

SEQ ID NO: 2 - protein, 9 aa, synthetic construct
  source 1..9: note = synthetic
REFPFYGDY

SEQ ID NO: 3 - protein, 6 aa, synthetic construct
  source 1..6: note = synthetic
EKHHSH

SEQ ID NO: 4 - protein, 38 aa, synthetic construct
  source 1..38: note = synthetic
DSHEKRHHGYRRKFHEKHHSHREFPFYGDYGSNYLYDN

SEQ ID NO: 5 - protein, 27 aa, synthetic construct
  source 1..27: note = synthetic
RKFHEKHHSHREFPFYGDYGSNYLYDN

SEQ ID NO: 6 - protein, 32 aa, synthetic construct
  source 1..32: note = synthetic
DSHAKRHHGYKRKFHEKHHSHRGYRSNYLYDN

SEQ ID NO: 7 - protein, 15 aa, synthetic construct
  source 1..15: note = synthetic
EKHHSHREFPFYGDY

SEQ ID NO: 8 - protein, 17 aa, synthetic construct
  source 1..17: note = synthetic
REFPFYGDYGSNYLYDN

SEQ ID NO: 9 - protein, 19 aa, synthetic construct
  source 1..19: note = synthetic
SHREFPFYGDYGSNYLYDN

SEQ ID NO: 10 - protein, 21 aa, synthetic construct
  source 1..21: note = synthetic
HHSHREFPFYGDYGSNYLYDN

SEQ ID NO: 11 - protein, 23 aa, synthetic construct
  source 1..23: note = synthetic
EKHHSHREFPFYGDYGSNYLYDN

SEQ ID NO: 12 - protein, 25 aa, synthetic construct
  source 1..25: note = synthetic
FHEKHHSHREFPFYGDYGSNYLYDN

SEQ ID NO: 13 - protein, 19 aa, synthetic construct
  source 1..19: note = synthetic
RKFHEKHHSHREFPFYGDY

SEQ ID NO: 14 - protein, 15 aa, synthetic construct
  source 1..15: note = synthetic
EKHHSHREFPFYGDY

SEQ ID NO: 15 - protein, 21 aa, synthetic construct
  source 1..21: note = synthetic
RKFHEKHHSHREFPFYGDYGS

SEQ ID NO: 16 - protein, 17 aa, synthetic construct
  source 1..17: note = synthetic
EKHHSHREFPFYGDYGS

SEQ ID NO: 17 - protein, 23 aa, synthetic construct
  source 1..23: note = synthetic
RKFHEKHHSHREFPFYGDYGSNY

SEQ ID NO: 18 - protein, 19 aa, synthetic construct
  source 1..19: note = synthetic
EKHHSHREFPFYGDYGSNY

SEQ ID NO: 19 - protein, 25 aa, synthetic construct
  source 1..25: note = synthetic
RKFHEKHHSHREFPFYGDYGSNYLY

SEQ ID NO: 20 - protein, 21 aa, synthetic construct
  source 1..21: note = synthetic
EKHHSHREFPFYGDYGSNYLY

SEQ ID NO: 21 - protein, 17 aa, synthetic construct
  source 1..17: note = synthetic
RKFHEKHHSHREFPFYG

SEQ ID NO: 22 - protein, 13 aa, synthetic construct
  source 1..13: note = synthetic
EKHHSHREFPFYG

SEQ ID NO: 23 - protein, 19 aa, synthetic construct
  source 1..19: note = synthetic
HHSHREFPFYGDYGSNYLY

SEQ ID NO: 24 - protein, 17 aa, synthetic construct
  source 1..17: note = synthetic
SHREFPFYGDYGSNYLY

SEQ ID NO: 25 - protein, 15 aa, synthetic construct
  source 1..15: note = synthetic
SHREFPFYGDYGSNY

SEQ ID NO: 26 - protein, 15 aa, synthetic construct
  source 1..15: note = synthetic
FPFYGDYGSNYLYDN

SEQ ID NO: 27 - protein, 13 aa, synthetic construct
  source 1..13: note = synthetic
FYGDYGSNYLYDN

SEQ ID NO: 28 - protein, 15 aa, synthetic construct
  source 1..15: note = synthetic
HHSHREFPFYGDYGS

SEQ ID NO: 29 - protein, 13 aa, synthetic construct
  source 1..13: note = synthetic
SHREFPFYGDYGS

SEQ ID NO: 30 - protein, 24 aa, synthetic construct
  source 1..24: note = synthetic
DSHAKRHHGYKRKFHEKHHSHRGY

SEQ ID NO: 31 - protein, 14 aa, synthetic construct
  source 1..14: note = synthetic
GYKRKFHEKHHSHR

SEQ ID NO: 32 - protein, 11 aa, synthetic construct
  source 1..11: note = synthetic
SHREFPFYGDY

SEQ ID NO: 33 - protein, 44 aa, synthetic construct
  source 1..44: note = synthetic
GGDSHEKRHHGYRRKFHEKHHSHREFPFYGDYGSNYLYDNLPET

(19) United States
(12) Patent Application Publication
(10) Pub. No.: US 2026/0295005 A1
(43) Pub. Date: Oct. 1, 2026

(54) HISTATIN FOR CORNEAL WOUND HEALING AND OCULAR SURFACE DISEASE

(71) Applicant: Visus Therapeutics, Inc., Seattle, WA (US)

(72) Inventors: Robert P. SAMBURSKY, Lakewood Ranch, FL (US); Robert W. VANDINE, Montoursville, PA (US); Peter Condon, Tierre Verde, FL (US)

(21) Appl. No.: 19/325,196

(22) Filed: Sep. 10, 2025

Related U.S. Application Data
(63) Continuation of application No. 19/029,568, filed on Jan. 17, 2025, now abandoned.
Application No. 19/029,568 is a continuation of application No. 18/917,448, filed on Oct. 16, 2024, now abandoned.
Application No. 18/917,448 is a continuation of application No. 18/061,699, filed on Dec. 5, 2022, now abandoned.
Application No. 18/061,699 is a continuation of application No. 16/525,367, filed on Jul. 29, 2019, now abandoned.
Application No. 16/525,367 is a continuation of application No. 15/180,476, filed on Jun. 13, 2016, now Pat. No. 10,413,587.
Application No. 15/180,476 is a continuation of application No. 13/788,803, filed on Mar. 7, 2013, now abandoned.
(60) Provisional application No. 61/648,845, filed on May 18, 2012.

(51) Int. Cl. A61K 38/17; A61K 9/00; A61K 38/08; A61K 38/10

(52) U.S. Cl. CPC A61K 38/1709; A61K 9/0048; A61K 9/0051; A61K 38/08; A61K 38/10

(57) ABSTRACT

Histatins may be used for corneal wound healing and as a treatment for ocular surface disease in humans and other animals. For example, histatins could be included in eye drops, eye gels, ointment, glue, or embedded in (polymer) contact lenses.

### REFERENCE TO SEQUENCE LISTING SUBMITTED ELECTRONICALLY VIA EFS-WEB

The content of the electronically submitted sequence listing in the .XML file (Name: 4902_0070009_SeqListing_ST26.xml; Size: 35,645 bytes; Date of Creation: May 24, 2023), filed with the application, is incorporated herein by reference in its entirety.

### REFERENCE TO RELATED APPLICATIONS

This is a continuation application of application Ser. No. 19/029,568, filed Jan. 17, 2025, which is a continuation application of application Ser. No. 18/917,448, filed Oct. 16, 2024 (now abandoned), which is a continuation application of application Ser. No. 18/061,699, filed Dec. 5, 2022 (now abandoned), which is a continuation application of application Ser. No. 16/525,367, filed Jul. 29, 2019 (now abandoned), which is a continuation application of application Ser. No. 15/180,476, filed Jun. 13, 2016 (now U.S. Pat. No. 10,413,587), which is a continuation application of application Ser. No. 13/788,803, filed Mar. 7, 2013 (now abandoned), which claims priority to Provisional Application No. 61/648,845, filed May 18, 2012, entitled “HISTATIN FOR CORNEAL WOUND HEALING AND OCULAR SURFACE DISEASE”. The benefit under 35 USC § 119 (e) of the United States provisional application is hereby claimed, and the aforementioned applications are hereby incorporated herein by reference.

### BACKGROUND OF THE INVENTION

### Field of the Invention

The invention pertains to the field of wound and disease healing. More particularly, the invention pertains to corneal wound healing and treating ocular surface disease using histatins.

### Description of Related Art

Histatins have been shown in in vitro studies to be wound healing agents from saliva. More specifically, WO 2009/087117 (and its US equivalent US Patent Publication 2011/0178010), herein incorporated by reference, identified peptides of histatin, which had wound healing properties in vitro.

Histatin 1 (Hst-1) and Histatin 2 (Hst-2) have been identified as major wound-closing factors in human saliva (“Discovery of the Wound Healing Capacity of Salivary Histatins”, thesis of Menno Johannes Oudhoff, Academic Centre for Dentistry Amsterdam (ACTA), VU University Amsterdam and University of Amsterdam, The Netherlands, 2010, herein incorporated by reference). These studies were all done in vitro and can not be translated to a finding for therapeutic or clinical use, especially since wound and disease healing are complex processes that need to be highly regulated in order to function properly.

### SUMMARY OF THE INVENTION

Histatins may be used for corneal wound healing and as a treatment for ocular surface disease in humans and other animals. For example, histatins could be included in eye drops, eye gels, ointment, glue, or embedded in (polymer) contact lenses.

In one preferred embodiment, a method of treating corneal wounds includes the step of administering a therapeutic amount of at least a peptide fragment of a histatin at a site of a corneal wound. The histatin is preferably administered using eye drops, gels, ointments including histatin, tissue glue, or by incorporating histatin into a contact lens worn by a patient. In a preferred embodiment, the therapeutic amount of histatin accelerates wound healing compared to corneal wounds not treated with histatin. The peptide fragment of the histatin preferably includes a sequence selected from the group consisting of: SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; and any combination of SEQ ID NO: 1 through SEQ ID NO: 33.

In some preferred embodiments, the histatin includes a) at least a peptide fragment of histatin 5 and b) at least a peptide fragment of histatin 1, at least a peptide fragment of histatin 2 or a combination of at least a peptide fragment of histatin 1 and at least a peptide fragment of histatin 2. In other preferred embodiments, the histatin is at least a peptide fragment of histatin 1, at least a peptide fragment of histatin 2, at least a peptide fragment of histatin 5, or any combination of a peptide fragment of histatin 1, a peptide fragment of histatin 2 and a peptide fragment of histatin 5.

In other preferred embodiments, the histatin is histatin 1 (SEQ ID NO: 4), histatin 2 (SEQ ID NO: 5), histatin 5 (SEQ ID NO: 30), or any combination of histatin 1, histatin 2, and histatin 5. In other preferred embodiments, the histatin includes a) histatin 5 (SEQ ID NO: 30) and b) histatin 1 (SEQ ID NO: 4), histatin 2 (SEQ ID NO: 5), or a combination of histatin 1 and histatin 2.

In another preferred embodiment, a therapeutic amount of at least a peptide fragment of a histatin is administered to an ocular surface to treat ocular surface disease. The histatin is preferably administered using eye drops, gels, ointments including histatin, tissue glue, or by incorporating histatin into a contact lens worn by a patient. In a preferred embodiment, the therapeutic amount of histatin accelerates healing of ocular surface disease compared to ocular surface disease not treated with histatin. The peptide fragment of the histatin preferably includes a sequence selected from the group consisting of: SEQ ID NO: 1; SEQ ID NO: 2; SEQ ID NO: 3; SEQ ID NO: 4; SEQ ID NO: 5; SEQ ID NO: 6; SEQ ID NO: 7; SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16; SEQ ID NO: 17; SEQ ID NO: 18; SEQ ID NO: 19; SEQ ID NO: 20; SEQ ID NO: 21; SEQ ID NO: 22; SEQ ID NO: 23; SEQ ID NO: 24; SEQ ID NO: 25; SEQ ID NO: 26; SEQ ID NO: 27; SEQ ID NO: 28; SEQ ID NO: 29; SEQ ID NO: 30; SEQ ID NO: 31; SEQ ID NO: 32; SEQ ID NO: 33; and any combination of SEQ ID NO: 1 through SEQ ID NO: 33.

In some preferred embodiments, the histatin includes a) at least a peptide fragment of histatin 5 and b) at least a peptide fragment of histatin 1, at least a peptide fragment of histatin 2 or a combination of at least a peptide fragment of histatin 1. In other preferred embodiments, the histatin is at least a peptide fragment of histatin 1, at least a peptide fragment of histatin 2, at least a peptide fragment of histatin 5 or any combination of a peptide fragment of histatin 1, a peptide fragment of histatin 2 and a peptide fragment of histatin 5.

In other preferred embodiments, the histatin is histatin 1 (SEQ ID NO: 4), histatin 2 (SEQ ID NO: 5), histatin 5 (SEQ ID NO: 30), or any combination of histatin 1, histatin 2, and histatin 5. In other preferred embodiments the histatin includes a) histatin 5 (SEQ ID NO: 30) and b) histatin 1 (SEQ ID NO: 4), histatin 2 (SEQ ID NO: 5), or a combination of histatin 1 and histatin 2.

### DETAILED DESCRIPTION OF THE INVENTION

Histatins are naturally occurring oral peptides produced by humans and non-human primates that demonstrate direct anti-infective activity, potent anti-inflammatory properties, and stimulate epithelial wound healing in several tissue and organ culture systems. A research facility has developed a technique to isolate this natural substance, making it a potential topical treatment for wounds.

In a preferred embodiment, a peptide including at least one amino acid sequence of at least eight amino acids adjacently present in Histatin 1, 2, 3, and/or 5 is used to treat a corneal wound or ocular surface disease.

In one preferred embodiment, a method of treating corneal wounds includes the step of administering a therapeutic amount of at least a portion of a histatin peptide at a site of a corneal wound. In another preferred embodiment, a method of treating ocular surface disease includes the step of administering a therapeutic amount of at least a portion of a histatin peptide to an ocular surface. The ocular surface diseases may include, but are not limited to, dry eyes, corneal ulcerations and erosions, inflammatory and infectious keratitis and conjunctivitis, surgical interventions, and trauma.

The histatin is preferably administered using eye drops, gels, ointments including histatin, tissue glue, or by incorporating histatin into a contact lens worn by a patient. In a preferred embodiment, the therapeutic amount of histatin accelerates wound or ocular surface disease healing compared to corneal wounds or ocular surface diseases not treated with histatin.

In some preferred embodiments, the histatin concentration is between approximately 0.1 μg/ml and approximately 1000 mg/ml. In other preferred embodiments, the histatin concentration is between approximately 0.1 μg/ml and 10 μg/ml. In some preferred embodiments, the histatin concentration is greater than or equal to approximately 1 μM.

The administering step may be repeated multiple times per day and/or for a plurality of days. In one preferred embodiment, this step is repeated at least one time a day for a plurality of days. In another preferred embodiment, the step is repeated chronically at least one time a day. In some preferred embodiments, the step is repeated up to hourly for a plurality of days. In another preferred embodiment, the step is repeated at least two times a day for a plurality of days. In yet another preferred embodiment, the step is repeated at least three times a day for a plurality of days, for example for seven days. In another preferred embodiment, the step is repeated four times a day for five days.

In one preferred embodiment, the histatin is a peptide including 8 to 44 amino acids. In some preferred embodiments, the peptide is a L-peptide. In other preferred embodiments, the peptide is a cyclic peptide.

In some preferred embodiments, the amino acid sequence of the histatin peptide is one or more of SEQ ID NOS: 1 through 33, or any combinations of these sequences. In alternative embodiments, one or more of the amino acid sequences have a substitution, deletion and/or insertion of up to 3 amino acids. In other alternative embodiments, one or more of the amino acid sequences have a substitution, a deletion and/or an insertion of two or less amino acids. In other alternative embodiments, one or more of the amino acid sequences have a substitution, a deletion, and/or an insertion in one amino acid.

The SEQ ID NO: 4 peptide is also known as Histatin 1 (Hst-1). Note that the first serine in this amino acid sequence may be a phosphoserine. The SEQ ID NO: 5 peptide is also known as Histatin 2 (Hst-2, also equivalent to amino acids 12-38 of Hst-1). The SEQ ID NO: 6 peptide is also known as Histatin 3 (Hst-3). The SEQ ID NO: 30 peptide is also known as Histatin 5 (Hst-5). Parts and fragments of each of these amino acid sequences may be used, alone or in combination, including but not limited to SEQ ID NOS: 1-3, 7-29 (for Histatin 1, Histatin 2 and Histatin 3) and SEQ ID NO: 31 (for Histatin 5) to facilitate wound closure in the embodiments described herein. While the L stereoisomer of the amino acids is preferred for the amino acid sequences described herein, D stereoisomers may alternatively be used. Alternatively, amino acid sequences that include these histatins and other amino acids, for example SEQ ID NO: 33, which is a sortase cyclized histatin (including all of Histatin 1), may be used in the embodiments described herein. Any histatin sequences could be cyclized and used in the embodiments described herein.

Some preferred embodiments use amino acid sequences from Hst-1 and/or Hst-2 in combination with amino acid sequences from Hst-5 to treat corneal wounds or ocular surface disease. In these embodiments, one or more amino acid sequences from Hst-1 and/or Hst-2 are chosen, and one or more amino acid sequences from Hst-5 are chosen. In some embodiments, the full length Histatin 1 (SEQ ID NO: 4), full length Histatin 2 (SEQ ID NO: 5), and/or the full length Histatin 5 (SEQ ID NO: 30) could be used. In other embodiments, portions of Hst-1, Hst-2, and/or Hst-5 could be used. For example, SEQ ID NO: 29, which is equivalent to amino acids 20-32 of Histatin 1, may be a preferred amino acid sequence to use for wound closure in some embodiments. In other examples, peptides including SEQ ID NO: 32, a peptide fragment of Histatin 1 and Histatin 2 that appears to be a core motif for wound closure, may be used. Other preferred sequences from Hst-1 and Hst-2 include, but are not limited to, SEQ ID NO: 8, SEQ ID NO: 9 and SEQ ID NO: 13. As another example, SEQ ID NO: 31, a fragment of Histatin 5 (Gusman et al., “Salivary Histatin 5 is an inhibitor of Both Host and Bacterial Enzymes Implicated in Periodontal Disease”, Infect. Immun. 2001, 69 (3): 1402, pp. 1402-1408, herein incorporated by reference), may be used, preferably in combination with Histatin 1 or Histatin 2 or fragments thereof. In other preferred embodiments, fragments of Hst-1 or Hst-2 are used with full length Hst-5 (SEQ ID NO: 30) or full length Hst-1 (SEQ ID NO: 4) or Hst-2 (SEQ ID NO: 5) are used with fragments of Hst-5 (for example, SEQ ID NO: 31). In yet other embodiments, any combination of fragments of Hst-1 and/or Hst-2, full length Hst-1 and/or Hst-2, fragments of Hst-5, or full length Hst-5 may be used. In some preferred embodiments, the concentration of the Hst-5 peptide used is greater than or equal to approximately 1 μM.

The amino acids and the peptides described herein may include at least one functional grouping (for example, an amine and/or carboxylic group) protected with a protective grouping in some embodiments. Since the peptides are applied to tissue, skin or a wound, a protected form of the peptide may be preferred to resist degradation. The form of protection needs to be biologically compatible and compatible with pharmaceutical use. Some examples include, but are not limited to, the acylation or the acetylation of the amino-terminal ends, cyclization or the amidation or the esterfication of the carboxy-terminal ends. Thus, the peptides described herein may be used in a protected form.

The peptides described herein may be made by traditional chemical synthesis, enzymatic synthesis, or any other method known in the art.

The peptides preferably include at least 8 amino acids. In one preferred embodiment, the peptides include a range of 8 to 44 amino acids, but the peptides may alternatively include more than 44 amino acids.

### In Vivo Studies

Efficacy studies of histatin for corneal wound healing utilize an animal model, namely rabbits. Histatins are naturally produced substances that stimulate healing in several tissue and organ culture systems. The results of these studies demonstrate that histatin has a significant dose dependent accelerated healing activity for corneal wounds.

Ocular surface disorders including, but not limited to, dry eyes, corneal ulcerations and erosions, inflammatory and infectious keratitis and conjunctivitis, surgical interventions, and trauma all lead to disruptions in the integrity of the corneal and conjunctival cellular barrier that result in increased risk of infection, pain, and reduced visual acuity. Histatins have a potential use in the treatment of ocular surface trauma/injury and infectious disease.

The outer layer of the cornea, the corneal epithelium, serves as a physical barrier against the environment and thus also as a line of defense to prevent infectious and/or toxic agents from infecting/affecting the tissue. When injury occurs to the surface of the cornea, the corneal epithelium spearheads a wound healing process (see, for example, Klyce S D, Crosson C E. “Transport processes across the rabbit corneal epithelium: a review”. Curr Eye Res. 1985; 4:323-331 and Lu L, Wang L, Shell B. “UV-induced signaling pathways associated with corneal epithelial cell apoptosis”. Invest Ophthalmol Vis Sci. 2003; 44:5102-5109, both herein incorporated by reference).

The study disclosed herein evaluates and quantifies the wound healing effects of histatins on the ocular surface of New Zealand White rabbits.

### Brief Methodology:

Epithelial defects are created in the right eye (oculus dexter, OD) of 12 New Zealand White rabbits. Due to animal regulations, bilateral wounding is not permitted. After the epithelial defects are made, the rabbits are randomized into treatment groups. Two (2) groups are treated with different cyclized histatin concentrations: 0.1 μg/ml and 10 μg/ml dissolved into an ophthalmic artificial tear preparation and delivered to the rabbits as an eye drop three times daily. Histatins known in the art, including, but not limited to, amino acid SEQ ID NOS: 1 through 33, which include Hst-1 (SEQ ID NO: 4), Hst-2 (SEQ ID NO: 5), Hst-3 (SEQ ID NO: 6), Hst-5 (SEQ ID NO: 30), and sortase cyclized histatin (SEQ ID NO: 33), may be used in these studies or in treatment protocols. One (1) group is treated with an inactive/inert formulation (control). This control group should receive the same vehicle identical to the other two groups but without histatin. An over-the-counter artificial tear preferably serves as the vehicle. The initial study included four (4) animals/group in three (3) groups, for a total of twelve rabbits.

The groups are to be treated with agent (either histatin or an inactive/inert formulation of artificial tears) three times/day (TID) for 7 days. Each rabbit group is preferably given moxifloxicin treatment to prevent infection.

The corneal wounds are then evaluated daily for the corneal wound healing abilities of histatin via fluorescein staining, fluorescent slit lamp biomicrophotography and computerized area determination. Evaluators are masked to the therapeutic treatment given to the rabbits. After healing, two (2) animals from each group are euthanized, and the corneas collected for histological processing (H&E staining with subsequent evaluation by veterinary histopathologist). The decision to perform histopathologic analysis after tissue procurement is made only if there is proven difference in healing between the different treatment groups and the controls. At study termination (study preferably continues for seven days), the remaining animals are euthanized.

### Results

The data from a first study using the methodology above is shown in Table 1. The histatin used in this study was cyclized histatin 1. The histatin 1 used was a sortase cyclized histatin with amino acid SEQ ID NO: 33, in which the “C-terminal” T is linked to the “N-terminal” G. Table 2 shows the mm size values (without the standard deviation) as an approximate percentage of the size at 1 hour post wound for each of the three groups. Pathological analysis of the rabbit corneas showed no toxicity.

TABLE 1

| Hours Post | Control | 0.1 μg/ml | 10 μg/ml |
|---|---|---|---|
| Wound | (mm²) | (mm²) | (mm²) |
| 0 | 69.3 ± 17.3 | 112.7 ± 37.8 | 88.4 ± 31.6 |
| 6 | 72.6 ± 15.2 | 96.2 ± 13.6 | 74.0 ± 14.7 |
| 24 | 50.6 ± 16.2 | 77.5 ± 25.0 | 51.4 ± 9.3 |
| 30 | 46.6 ± 25.8 | 48.8 ± 8.0 | 37.7 ± 9.4 |
| 48 | 13.6 ± 20.2 | 2.7 ± 3.1 | 0.00 ± 0.07 |
| 54 | 5.2 ± 7.9 | 0.00 ± 0.00 | 0.00 ± 0.00 |
| 72 | 0.00 ± 0.00 | 0.00 ± 0.00 | 0.00 ± 0.00 |

TABLE 2

| | Control- | 0.1 ug/ml- | 10 ug/ml- |
|---|---|---|---|
| | Percentage | Percentage | Percentage |
| Hours | of 1 hour | of 1 hour | of 1 hour |
| Post | post | post | post |
| Wound | wound size | wound size | wound size |
| 0 | 100% | 100% | 100% |
| 6 | 105% | 85% | 84% |
| 24 | 73% | 69% | 58% |
| 30 | 67% | 43% | 43% |
| 48 | 20% | 2% | 0% |
| 54 | 8% | 0% | 0% |
| 72 | 0% | 0% | 0% |

The results above show that histatin demonstrates a significant dose dependent accelerated healing activity of corneal wounds. While Table 2 does not take into account the standard deviations from Table 1, the percentages clearly indicate that the wounds treated with 0.1 μg/ml or 10 μg/ml histatin healed faster (shrunk more) at each of the time points where data was collected. These results are the first results of their kind done using in vivo animal studies.

Histatins and peptide portions or peptide fragments of histatins may be used to accelerate corneal wound healing or ocular surface disease healing in humans and other animals. In preferred embodiments, histatin 1 (Hst-1), histatin 2 (Hst-2), histatin 5 (Hst-5), peptide fragments of Hst1, Hst2, or Hst5, or any combinations thereof may be used. In other embodiments, histatin 3 (Hst-3) or the D-enantiomer of histatin 2 (D-Hst-2), or peptide fragments thereof, may be used. Any combinations of any of the histatins may be used. In preferred embodiments, histatin concentrations between 0.1 μg/ml and 1000 mg/ml may be used. Peptides with amino acid SEQ ID NOS: 1-33, histatins known in the art, the peptides disclosed in WO 2009/087117 or the peptides disclosed in Dr. Menno Johannes Oudhoff's thesis, “Discovery of the Wound-Healing Capacity of Salivary Histatins”, 2010, department of Oral Biochemistry of the Academic Centre for Dentistry Amsterdam (ACTA), VU University Amsterdam and University of Amsterdam, The Netherlands, herein incorporated by reference, may be used.

In one preferred embodiment, histatin 1 (Hst-1) or histatin 2 (Hst-2) in combination with histatin 5 (Hst-5), peptide fragments of Hst-1 or Hst-2 in combination with peptide fragments of Hst-5, or any combination, are used. Hst-5 inhibits production of Matrix Metalloproteases (MMPs).

The combination of the Hst-1/Hst-2 healing properties with the Hst-5 inhibiting MMPs should be very effective. In some preferred embodiments, a concentration of at least approximately 1 μM of Hst-5, or a fragment of Hst-5, is used.

Histatins could be administered to humans or other animals with a corneal wound or ocular surface disorders. Some methods of administration include, but are not limited to, incorporating the histatin into eye drops, gels or ointments, incorporating the histatin into tissue glue used to transiently seal corneal injuries, or embedding the histatin into (polymer) contact lenses.

The histatins may be administered in any combination of daily treatments for any number of days in order to produce therapeutic results. In one preferred embodiment, the histatin is administered at least once a day for a plurality of days. In another preferred embodiment, the histatin is administered at least once a day chronically (for an extended period of time). In another preferred embodiment, the step may be repeated two, three, four, five times or more, or hourly, for a plurality of days or chronically. In one example, the histatin is repeated three times a day for seven days. In another example, histatin is administered four times a day for five days.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

1-20. (canceled)

21. An ophthalmic formulation comprising:
about 0.1 μg/ml to about 1000 mg/ml of a first peptide comprising histatin 1 or histatin 2, wherein the first peptide comprises a sequence selected from the group consisting of SEQ ID NO: 4; SEQ ID NO: 5; SEQ ID NO: 8; SEQ ID NO: 9; SEQ ID NO: 13; SEQ ID NO: 29; and SEQ ID NO: 33 and the sequence may optionally include a substitution, deletion and/or insertion of up to 3 amino acids; and
about 0.1 μg/ml to about 1000 mg/ml of a second peptide comprising histatin 5, wherein the second peptide comprises a sequence selected from SEQ ID NO: 30 and SEQ ID NO: 31 and the sequence may optionally include a substitution, deletion and/or insertion of up to 3 amino acids;
wherein the ophthalmic formulation accelerates healing of ocular surface disease compared to healing of ocular surface disease not treated with the ophthalmic formulation.

22. The ophthalmic formulation of claim 21, wherein the concentration of each of the first peptide and the second peptide in the composition is about 0.1 μg/ml to about 10 μg/ml.

23. The ophthalmic formulation of claim 21, wherein the ophthalmic formulation is configured for administration to an ocular surface at least one time per day for a plurality of days.

24. The ophthalmic formulation of claim 21, wherein the ophthalmic formulation is configured for administration to an ocular surface at least one time a day.

25. The ophthalmic formulation of claim 21, wherein the amino acid sequence of the first peptide is selected from the group consisting of SEQ ID NO: 4; SEQ ID NO: 5; SEQ ID NO: 29; and SEQ ID NO: 33.

26. The ophthalmic formulation of claim 21, wherein the ocular surface disease is selected from the group consisting of dry eyes, corneal ulcerations and erosions, inflammatory and infectious keratitis and conjunctivitis, surgical interventions, and trauma.

27. The ophthalmic formulation of claim 21, wherein one or both of the first peptide and the second peptide are each independently 8 to 44 amino acids in length.

28. The ophthalmic formulation of claim 21, wherein the first peptide and the second peptide are L-peptides.

29. The ophthalmic formulation of claim 21, wherein the first peptide is a cyclic peptide.

30. The ophthalmic formulation of claim 29, wherein the first peptide comprises SEQ ID NO: 33.

31. The ophthalmic formulation of claim 21, wherein the first peptide comprises SEQ ID NO: 33.

32. The ophthalmic formulation of claim 21, wherein the ophthalmic formulation is an eye drop, gel, ointment, or tissue glue.

33. The ophthalmic formulation of claim 21, wherein the ophthalmic formulation is configured for administration to an ocular surface up to hourly for a plurality of days.

34. The ophthalmic formulation of claim 29, wherein the cyclic peptide is a sortase cyclized peptide.

35. The ophthalmic formulation of claim 21, wherein the first peptide, the second peptide, or both comprises at least one protective grouping.

36. The ophthalmic formulation of claim 35, wherein the at least one protective grouping is selected from an amine, a carboxylic group, an acylation, and acetylation, a cyclization, an amidation, and an esterification.

37. The ophthalmic formulation of claim 21, wherein the formulation is configured for administration to an open wound.

38. The ophthalmic formulation of claim 21, wherein the ophthalmic formulation is further configured for coadministration with an antibiotic.

39. The ophthalmic formulation of claim 21, wherein the ophthalmic formulation is incorporated into a contact lens.

40. The ophthalmic formulation of claim 32, wherein the ophthalmic formulation is an eye drop.
